(19) United States
(12) Patent Application Publication
(10) Pub. No.: US 2018/0325461 A1
(43) Pub. Date: Nov. 15, 2018

(54) Systems and Methods for Producing Quantitatively Calibrated Grayscale Values in Magnetic Resonance Images

(71) Applicant: Northwestern University, Evanston, IL (US)

(72) Inventors: TIMOTHY J. CARROLL, CHICAGO, IL (US); YONG I. JEONG, CARY, IL (US); DAVID MANGLANO, PALOS PARK, IL (US); PATTANANASAK MONGKOLWAT, CHICAGO, IL (US); THOMAS GALLAGHER, Golden, CO (US); CHARLES CANTRELL, CHICAGO, IL (US)

(21) Appl. No.: 15/577,526

(22) PCT Filed: May 27, 2016
(86) PCT No.: PCT/US16/34661

§ 371 Date: Nov. 28, 2017

Related U.S. Application Data
(60) Provisional application No. 62/168,307, filed on May 29, 2015.

(51) Int. Cl. A61B 5/00; G01R 33/58; G01R 33/56; G06F 19/24; G16H 30/40

(52) U.S. Cl. CPC A61B 5/7267; G01R 33/58; G01R 33/28; G06F 19/24; G16H 30/40; G01R 33/5608

(57) ABSTRACT

Systems and methods for converting grayscale values in magnetic resonance images having different image contrasts into normalized estimations of physiological quantities are provided. These methods provide consistency of data from scan-to-scan, which allows the data to be used for high-fidelity radiogenomic analyses. Imaging-derived radiogenomic heat maps, based on trained models of quantitative radiogenomic associations, can be generated from, the normalized images and can provide a novel technique to survey the often varied genetic landscape within a tumor.

### CROSS-REFERENCE TO RELATED APPLICATIONS

This application claims the benefit of U.S. Provisional Patent Application Ser. No. 62/168,307, filed on May 29, 2015, and entitled “SYSTEMS AND METHODS FOR PRODUCING QUANTITATIVELY CALIBRATED GRAYSCALE VALUES IN MAGNETIC RESONANCE IMAGES.”

### STATEMENT REGARDING FEDERALLY SPONSORED RESEARCH

This invention was made with government support under EB017928 awarded by the National Institutes of Health. The government has certain rights in the invention.

### BACKGROUND OF THE INVENTION

The field of the invention is systems and methods for magnetic resonance imaging (“MRI”). More particularly, the invention relates to systems and methods for calibrating magnetic resonance images to produce standardized and normalized quantitative images of physiological parameters.

Approximately 22,400 people will be diagnosed with gliomas each year, and of those approximately 12,000 will be glioblastoma multiforme (“GBM”), which is the most common and deadly primary CNS malignancy. Despite standard therapies, GBMs have a two year survival rate of only 27%. Some of the challenges in designing a treatment plan for brain tumor patients, and essentially all cancer patients, is selecting which patients will do well on a certain treatment regimen and which will do poorly. It is now understood that the often-variable genetic landscape within tumors can dictate a response to therapy.

A confounding treatment effect in patients on the Stupp regimen mimicked early tumor recurrence on MRI, and was appropriately termed pseudoprogression. Interestingly, pseudoprogression is much more common in a distinct class of GBM featuring a unique epigenetic modification: methylation of the promoter site of 06-methylguanine-DNA methyltransferase (“pMGMT”). Methylated MGMT (“pMGMT+”) tumors lack ability to fix damage wrought by alkylating agents such as temozolomide, which confers better prognoses due to increased sensitivity to the drug. Selection or modification of therapy based on a genomic trait such as pMGMT status is a step toward individualized GBM treatment. A strong movement to sophisticate treatment and classification of many cancers, including GBM, based on their genetic underpinnings has now taken hold.

Currently, only invasive methods such as biopsy and resection are available to assess the genetic profile in a tumor. Considering that not all tumors or patients are readily operable, there remains a need to provide non-invasive techniques for assessing a genetic profile in patients.

The discipline of radiogenomics aims to establish MRI imaging features that can noninvasively report the underlying genetic profile in a tumor. The underlying principle is that the local genetic alterations in tumors result in protein expression that dictates microscopic and macroscopic tissue properties and physiology, and directly modulates an appearance on MRI. Important radiogenomic investigations relevant to CNS tumors have attempted to link qualitative and quantitative MRI features, tumor volumes, geometry, MR perfusion characteristics, diffusion-weighted imaging (“DWI”), and diffusion tensor imaging (“DTI”) to various genetic alterations. For instance, MR perfusion metrics including cerebral blood volume (“rCBV”) have been linked to tumor grade, vascular endothelial growth factor receptor (“VEGFR”) amplification, and response to the drug bevicizumab.

Another notable target for radiogenomic association is the apparent diffusion coefficient (“ADC”) derived from diffusion-weighted images, which has been used to infer tumor cellularity, WHO-tumor grade, response to bevicizumab, and pMGMT methylation status.

The existence of the National Institutes of Health (“NIH”) databases including The Cancer Genome Atlas (“TCGA”) and The Cancer Imaging Archive (“TCIA”) underscores this initiative to centralize and foster such radiogenomic analyses. In the era of big data, however, many challenges surface when attempting to conglomerate and then analyze a large database, the most significant of which is lack of standardization of protocols and lack of intersubject image normalization.

While conventional MRI sequences (e.g., T1, T2, FLAIR, T1 post contrast, ADC) are still the current workhorse of tumor imaging, and while they are omnipresent in most institutions across the country, they are not all acquired in the same way or on the same scanner platform. MR image contrast for these sequences is a carefully chosen medley of repetition time (“TR”), echo time (“TE”), or inversion time (“IR”), which varies across institutions, across scanners, with magnetic field strengths, number of coil elements, and a host of other factors. Furthermore, receiver gains are iteratively tuned for optimal patient imaging on a scan-by-scan basis, which inevitably modulates the signal intensity in the acquired images to further refine diagnostic image quality, reduce noise, and mitigate artifacts. For a true, high fidelity radiogenomic analysis, and to apply an established radiogenomic link to a new or foreign MRI scan (i.e., one obtained with different parameters) a mechanism must exist to provide a universal normalization to the imaging datasets.

### SUMMARY OF THE INVENTION

The present invention overcomes the aforementioned drawbacks by providing a method for producing from magnetic resonance images, quantitative maps that indicate quantitative values of physiological parameters. The method generally includes providing a plurality of magnetic resonance images to a computer system, where these images depict a subject and have different physical contrasts. The plurality of magnetic resonance images are then converted with the computer system to produce quantitative physiological parameter maps by calibrating the magnetic resonance images based on signal models associated with how the magnetic resonance images were acquired.

In some aspects, a method for producing from magnetic resonance images, a genomic profile map that indicates a level of genetic expression in a tissue is also provided. A plurality of magnetic resonance images depicting a subject are provided to a computer system, and these plurality of magnetic resonance images have different physical contrasts (e.g., T1, T2, apparent diffusion coefficient). The plurality of magnetic resonance images are then converted to produce quantitative physiological parameter maps by calibrating the magnetic resonance images based on signal models associated with how the magnetic resonance images were acquired. A likelihood of a particular gene expression in a tissue in the subject is then determined based on the quantitative physiological parameter maps. A genomic profile map that indicates a level of genetic expression for the particular gene is then produced using the determined likelihood of the particular gene expression in the tissue.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a flowchart setting forth the steps of an example method for calibrating magnetic resonance images having different image contrasts to convert the qualitative information contained in the images to quantitative information of physiological parameters;

FIG. 2 is a flowchart setting forth the steps of an example method for producing a genomic profile map based on magnetic resonance images that have been calibrated using the quantitative calibration method described in FIG. 1;

FIG. 3 is a representative example, comparing ADC values before and after normalization across 21 patients, where it can be seen that the grayscale values have a wide variability, while the normalized ADC values are steady under a certain range;

FIGS. 4A-4E illustrate grayscale T1 post contrast (FIG. 4A), a normalized quantitative T1 (ms) map (FIG. 4B), a normalized quantitative T1 (ms) post contrast map (FIG. 4C), a normalized quantitative T2 (ms) map (FIG. 4D), and a normalized quantitative ADC (×10⁻³ mm²/s) map;

FIG. 5 illustrates an example genomic profile map indicating positive predictive value of tumor methylation (low=dark blue (pMGMT−), moderate=green (pMGMT+), high=red (pMGMT+)) within the same enhancing tumor, which was reported as pMGMT− negative due to sampling of the tumor periphery (thin arrow); and

FIG. 6 is a block diagram of an example computer system that can implement the methods described here.

### DETAILED DESCRIPTION OF THE INVENTION

Described here are systems and methods for calibrating multi-contrast magnetic resonance images across many different subjects and imaging platforms and to thereby convert the qualitative grayscale images into quantitative maps of physiological parameters. As one example, multi-contrast magnetic resonance images can include magnetic resonance images having different image contrasts (e.g., T1-weighting, T2-weighting, proton density weighting, diffusion weighting). This calibration procedure allows for more accurately quantifying parameters such as apparent diffusion coefficient (“ADC”), T1 values, and T2 values. Based on this calibration, true integration of many different image types into one large imaging database, across clinical sites, or across different MRI scanners can be achieved.

One advantage of this calibration technique is the ability to convert previously acquired, qualitative grayscale images into quantitative maps of physiological parameters. Moreover, the calibration process removes biases that may result from using different scanners; thus, the quantitative maps produced by the calibration method are platform-independent. Removing these biases allows for direct comparison of images across different clinical sites, scanners, or subject populations.

As one example, the normalized quantitative values derived from such magnetic resonance images can be utilized as a predictor of gene expression levels, such as the status of pMGMT in a particular subject. In addition, the calibration and normalization described here can provide for radiogenomic findings to be mapped to a parametric color map of local genomic predictions throughout the voxels corresponding to tumor.

The identification of gene expression levels inside a tumor is critical for the treatment of cancer. Currently, the genetic information is acquired by performing a biopsy and analyzing the cells via DNA microarray analysis. This method takes up a significant amount of time and is very invasive. The methodology described here, however, enables the use of various magnetic resonance scans and genomic information for model development, and utilizes noninvasive MRI to produce parametric maps of gene signatures. These maps provide the gene expression levels necessary to offer personalized therapy to patients without the cost and surgery normally associated with genetic evaluation of a tumor.

The systems and methods described here thus have the potential to reduce the number of biopsies for gene expression arrays of cancer patients. The systems and methods described here may also act as research tools for accelerating the research of cancer due to the reduction of time for analyzing gene expression signatures, and the ease of use compared to prior methods.

The calibration of signal intensities described here allows for direct comparison of images across a wide array of MRI scanners and scan protocols. As one benefit, this calibration allows advanced machine learning algorithms to be “trained” to detect aberrant local gene expression (i.e., genomic changes) that result from disease (e.g., cancer). These trained machine learning algorithms can, in turn, be used to produce parametric maps of local gene expressions within a tumor. As an example, these maps of gene expression can represent on a patient-by-patient basis distinct genetic phenotypes (single or multiple) within a tumor.

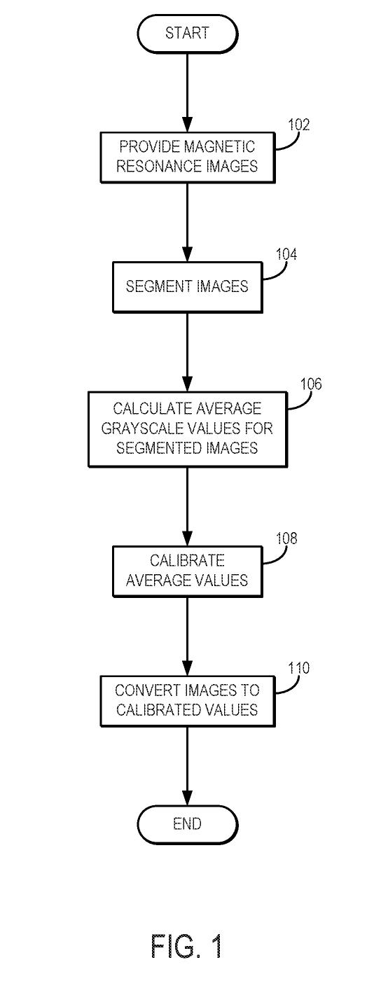

Referring now to FIG. 1, a flowchart is illustrated as setting forth an example of a method for calibrating magnetic resonance images, which may include cross-normalizing the images. The method includes providing magnetic resonance images to a computer system, as indicated at step 102. As one example, the magnetic resonance images can be provided by retrieving previously acquired images from a database. As another example, the magnetic resonance images can be provided by acquiring the images with an MRI system. The images are then normalized, as generally described below.

First, the magnetic resonance images are segmented into different tissue types, as indicated at step 106. For instance, when the images depict a brain the images can be segmented into white matter, gray matter, and cerebrospinal fluid. As one example, such segmentation can be performed using the Statistical Parametric Mapping software toolkit described by K. J. Friston, et al., in Statistical Parametric Mapping: The Analysis of Functional Brain Images; Elsevier, London; 2006. This segmentation process results in the creation of segmentation masks for the different segmented tissue types.

Using the segmented masks, average grayscale values for each tissue type are calculated, as indicated at step 108. These values are then calibrated against known standard values for the respective tissues, as indicated at step 110. For instance, the average values are calibrated against known standard values for the type of MRI scan (e.g., spin echo, gradient recalled echo, inversion recovery) used to acquire the original images. As one example, the average values are calibrated using an exponential fit to a signal model associated with the MRI scan type.

Applying the resulting fit to each corresponding type of MRI scan converts the grayscale values in the original images to normalized, quantitative values, as indicated at step 112. These quantitatively calibrated images can then be stored for later use or processing.

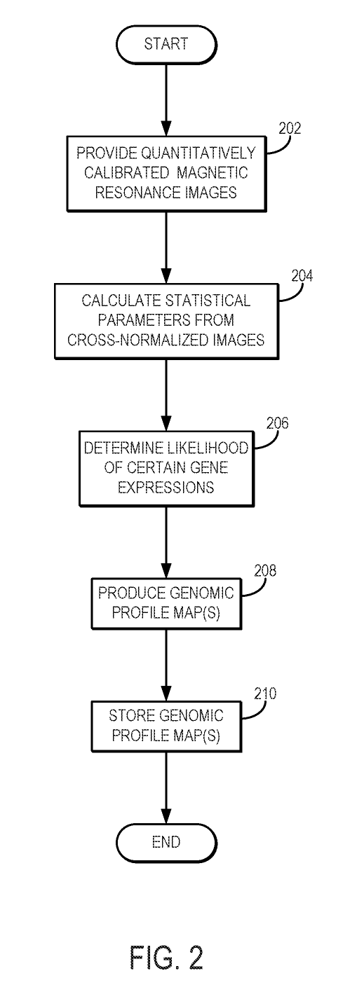

Referring now to FIG. 2, a flowchart is illustrated as setting forth the steps of an example method for producing a genomic profile map based on magnetic resonance images that have been calibrated using the quantitative calibration method described above. The method includes providing such quantitatively calibrated images, as indicated at step 202. Statistical parameters that describe the variability and distribution within the images are then calculated, as indicated at step 204.

The likelihood of a certain gene expression can then be determined, as indicated at step 206, by using machine learning methods, such as neural networks, that are trained based on quantitative parametric images and genetic information. As one example, the machine learning algorithm can be trained on relating quantitative magnetic resonance images with genetic information that was obtained from the same subjects depicted in those images. To this end, the calibration method described above can be used to help train the machine learning algorithm for those instances where qualitative, but not quantitative, images are available.

The model resulting from this machine learning is then applied to the quantitatively calibrated magnetic resonance images to produce parametric maps of gene expression levels, as indicated at step 208. These parametric maps may be referred to as genomic profile maps. These genomic profile maps can be viewed as a radiogenomic “heat map,” indicating the predictions of local gene profiles based on the quantitatively calibrated images. After the genomic profile maps are created, they are stored for display or further processing as indicated at step 210.

As one example application, the MRI-based genomic profile maps indicating local gene expression can be integrated into the nominal radiologic workflow to improve the imaging workup of cancers and other pathologies. This non-invasive review of local gene expression provides personalized (i.e., patient-specific) information that can supplement clinical decisions for treatment options, such as by providing evaluation of response to chemotherapy. For instance, the methods described here also provide information that can enable physicians to identify distinct genetic subtype (i.e., phenotypes) within a single tumor. Genetic phenotyping predicts response to therapy, thereby allowing for more targeted treatment and evaluation of treatment response.

It is contemplated that the imaging of local gene expression using the methods described here will provide a more precise estimation of adherent pathways when compared with gross resection or biopsy. Furthermore, compared to performing a biopsy to obtain genetic information, MRI is quick and noninvasive.

### Example: A Method for Displaying Local Gene Expression with MRI

A retrospective analysis was performed on magnetic resonance images and gene expression data for treatment naïve glioblastoma multiforme (“GBM”), provided by the database of The Cancer Genome Atlas (“TCGA”) and The Cancer Imaging Archive (“TCIA”). This database included 260 patients and 570 scans; however, not all cases could be included in the study because some scans lacked the necessary genomic information, or otherwise lacked images obtained with certain imaging sequences.

In this example study, radiogenomic analysis was conducted to investigate the relationship between quantitative T1, T2, and ADC values and the status of methylation of the O-16-methylguanine DNA methyltransferase promoter (“pMGMT”) in GBM, which has shown to predict sensitivity to alkylating chemotherapeutic agents, such as temozolomide. Quantitative values for T1, T2 and ADC images, both before and after the quantitative calibration described above, were studied in relation to microarray analysis of biopsy samples to assess the utility of MRI to indicate methylated (pMGMT+) and unmethylated (pMGMT−) tumor phenotypes.

TCIA is a repository for MRI scans from GBM patients that have been gathered from many different institutions, and given the potential variability in image acquisition parameters (or outright lack of this information), it exemplifies several major challenge discussed above, particularly when attempting investigate quantitative radiogenomic links. For instance, derivation of true quantitative ADC from the TCIA is often not possible, as only grayscale images are available. To address this issue, the method described above relates image grayscale pixel intensities from T1, T2, and ADC maps back to quantitative values. The quantitative calibration described above then allows for direct comparison of T1, T2, and ADC across subjects, which are unique, quantum properties of all tissues in the body, including a brain tumor or other pathologies.

If a quantitative radiogenomic link is discovered, and a classification model (e.g. linear regression, machine learning techniques) is proposed to predict the genetic profile based on the imaging trait, this normalization method provides a means to quickly prepare and introduce new MRI studies into the existing model. The output of such an analysis can be viewed as a radiogenomic heat map, indicating the predictions of local gene profiles based entirely on the normalized imaging.

Patient (Data) Selection.

In total, 21 patients were selected based on the availability of MR images of interest (T1, T2, and ADC) and the information of pMGMT methylation status. The types of MR images of main interest were the T1-weighted images (before and after gadolinium based contrast agent injection), T2-weighted images, and ADC images. The four types of images were collected based on a combination of the series description, TR, TE and contrast agent usage. The images, which were in DICOM format, were converted to the NIfTI (.nii) format using a third-party software Statistical Parametric Mapping (SPM Version 8 Wellcome Trust Centre for Neuroimaging, London, UK). Using SPM, the images were also co-registered and re-sliced, and white matter (WM), gray matter (GM) and cerebral spinal fluid (CSF) probability masks were segmented. Regions of interest (ROIs) of the tumor were drawn by neuroradiology fellows and doctors.

Genomic Processing.

In this example study, all of the pMGMT methylation status information was obtained from the TCGA-GBM database. This raw data was then processed to dichotomize it into binary values using algorithms presented by P. Bady, et al. in “MGMT methylation analysis of glioblastoma on the Infinium methylation BeadChip identifies two distinct CpG regions associated with gene silencing and outcome, yielding a prediction model for comparisons across datasets, tumor grades, and CIMP-status,” Acta Neuropathol, 2012; 124(4): 547-560. Analysis of GBM methylation status proceeded through the Illumina HumanMethylation450 platform (and, through legacy TCGA data, the Illumina HumanMethylation80 platform), in which sample DNA is reacted with bisulfite to deaminate unmethylated cytosine residues to uracil; methylated cytosine residues resist deamination. Fragmented sample libraries of bisulfite-treated DNA bind to engineered oligonucleotides attached to 3 micron silica beads with fluorescent dye (the Illumina BeadChip platform). Illumina software return a signal intensity as a β-value, which is then adjusted to an M-value to eliminate heteroscedasticity in statistical analysis of methylation probes:

$${M}_{i} = {log}_{2} ( \frac{β}{1 - β} ) . \qquad ( 1 )$$

Several hundred methylation sites may exist for a given gene, but not all methylation sites are predictive of methylation-dependent gene silencing. Stepwise logistic regression reveals methylation sites with the greatest impact on gene expression; such a model specifies two probes of significance for determining MGMT promoter site methylation in GBM.

Absolute Normalization of Image Contrast.

Grayscale pixel intensities were converted to normalized physical values using the methods described above. For example, as described above, this process includes using a linear regression between pixel intensity values and physical values of T1, T2, and ADC. Reference values of white matter and gray matter pixel intensity (i.e., grayscale values) values were used in separate regression analyses for each contrast weighting (T1, T2 and ADC). These standard values are listed in Table 1, which lists the average repetition time and echo time for each image type in addition to the standard white matter and gray matter values.

TABLE 1

| Image | | | | |
|---|---|---|---|---|
| Type | WM Reference | GM Reference | TR (ms) | TE (ms) |
| T1 | 790 ms | 920 ms | ~682 ms | ~15 ms |
| T2 | 75 ms | 85 ms | ~4962 ms | ~93 ms |
| ADC | 0.7 × 10⁻³ mm²/s | 0.89 × 10⁻³ mm²/s | ~5480 ms | ~93 ms |

For gadolinium enhanced T1-weighted images, the linear fit obtained from the T1 pre calibration was used. The linear regression was derived from a linear approximation of the MR signal equations for various pulse sequences by expanding the exponential functions as power series to the first order term.

The MRI physics of the pulse sequence used to acquire the MR image results in different signal equations that determine the parameterization used in the recursion analysis. Examples of these signal equations are shown in Table 2, which shows the signal equations and the linearly approximated forms for each type of pulse sequence. In Table 2, S is the signal, G is the gain, k is the coil sensitivity, ρ is the proton density, b is the b-value, A and B are constants, R₁ is the inverse of T₁, and R₂ is the inverse of T₂.

TABLE 2

| Sequence | | | | |
|---|---|---|---|---|
| Type | Signal Equation | T1-weighted | T2-weighted | ADC Map |
| Spin Echo | $S = Gk ρ ( 1 - {e}^{\frac{TR}{{T}_{1}}} ) {e}^{\frac{TE}{{T}_{2}}}$ | S = A + B · R₁ | S = A + B · R₂ | |
| Gradient Recalled Echo | $S = \frac{Gk ρ ( 1 - {e}^{\frac{TR}{{T}_{1}}} ) sin θ {e}^{\frac{TE}{{T}_{2}}}}{1 - cos θ {e}^{\frac{TR}{{T}_{1}}}}$ | $\frac{1}{S} = A + B \cdot {T}_{1}$ | S = A + B · R₂ | |
| Inversion Recovery | $S = Gk ρ ( 1 - 2 {e}^{\frac{T 1}{{T}_{1}}} + {e}^{\frac{TR}{{T}_{1}}} ) {e}^{\frac{TE}{{T}_{2}}}$ | S = A + B · R₁ | S = A + B · R₂ | |
| Diffusion | S = GS₀e^{-b ADC} | | | S = A + B · ADC |

The signal equations were then simplified depending on the type of image (i.e., T1-weighted image or T2-weighted image). For T1-weighted images, TE was significantly less than the T2 of the tissues of the brain, so the ê(−TE/T2) term was approximated as one. If the MR image was T2-weighted, then TR was set significantly greater than the T1 of the brain tissues; therefore, the ê(−TR/T1) term was approximated as zero. For scans that used an inversion recovery sequence, the ê(−TI/T1) term was approximated as zero as well because the inversion time (“TI”) was significantly greater than the T1 of brain tissues.

The remaining exponential functions were then reduced to the first order term of its power series in the form of 1+x. By taking this approach, all of the variables except T1, T2, ADC, and S were treated as constants. For T2-weighted images, the signal equations for the three types of pulse sequences were all reduced to S=A+B*R2 where R2 is the inverse of T2. For T1-weighted images, the signal equation for GRE pulse sequences was reduced to (1/S)=A+B*T1, while the signal equations for SE and IR pulse sequences were reduced to S=A+B*R1 where R1 is the inverse of T1. After the coefficients A and B were obtained from a linear fit for each type of image, they were applied to the corresponding images to convert the pixel intensity values to relative physical values.

In the TCIA database ADC images do not yield quantitative information, as the strength of the diffusion sensitizing gradients (B-values) were inadvertently removed from the DICOM header during de-identification. The scaling of ADC values to grayscale during the calculation of ADC dictate the use of S=A+B*ADC. Where A and B were fitted constants, S was grayscale and ADC were reference values.

After the fits were calculated, images of grayscale values were converted to the corresponding physical values T1, T2, and ADC, as described above.

Radiogenomic Image Analysis.

In this example study, ROIs were drawn by a trained physician and adjudicated by a CAQ board certified neuroradiologist based on un-normalized T1-weighted and T2-weighted images to mimic the clinical workflow. Readers were blinded to both gene expression results and absolution normalization of T1 and T2. ADC images were not used in the drawing of ROIs. Enhancing solid tumor was included in the ROI, however central necrosis was excluded.

Mean values of T1, T2, and ADC were compared between tumors deemed to be pMGMT+ and pMGMT− from genetic analysis. When imaging metrics were found to have statistically significant mean values, a Receiver Operator Characteristic (“ROC”) analysis was performed to determine the improvement in distinguishing pMGMT+ from pMGMT− and reported as the Area Under the Curve (“AUC”). Local variation in the ADC parameter (1/ADC) was then used to assign a scale to tissue. The true positive fraction (“TPF,” in percentage) that the MGMT promoter gene was methylated and parameterized was extracted from the ROC curve using the well-known Gaussian integral:

$${pMGMT}^{+} ( A D {C}^{- 1} ) = \frac{1}{2} ( 1 + erf ( \frac{A D {C}^{- 1} - μ}{\sqrt{2 σ}} ) ) ; \qquad ( 2 )$$

where μ and σ are one type of statistical parameter derived from the test of diagnostic accuracy, which in this example was the ROC analysis. This function allows for a standardized color scale to be used in the parametric display of methylation status within an individual tumor. The local genomic predictions can be reintroduced into the radiology workflow, and can be viewed as a voxel-wise color map throughout the tumor, overlaid on the anatomic T1 post contrast image.

Results.

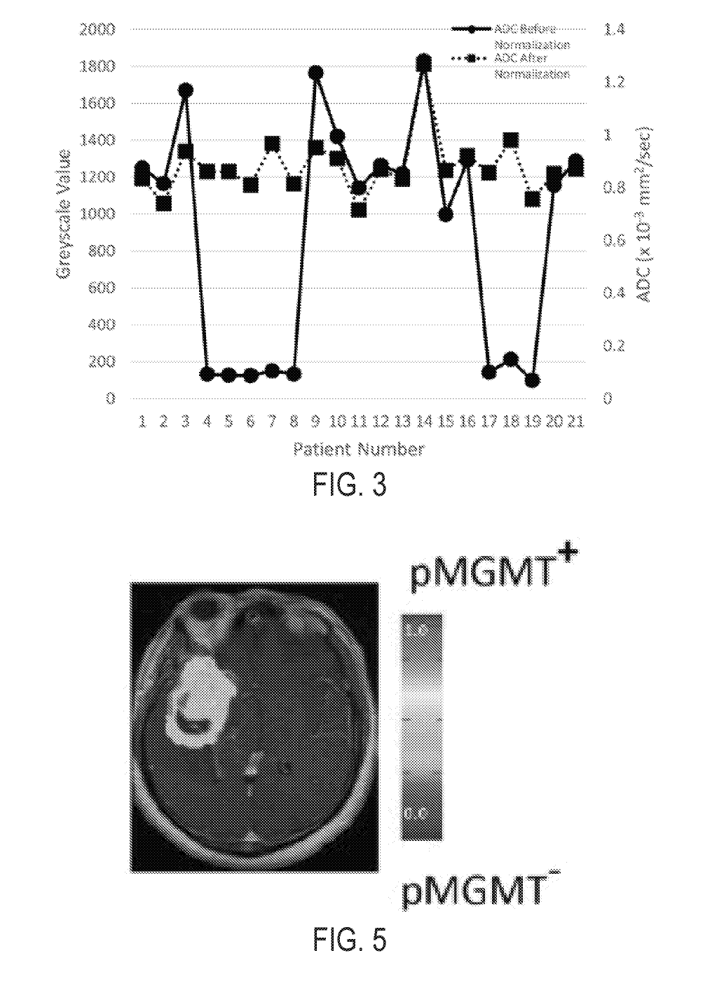

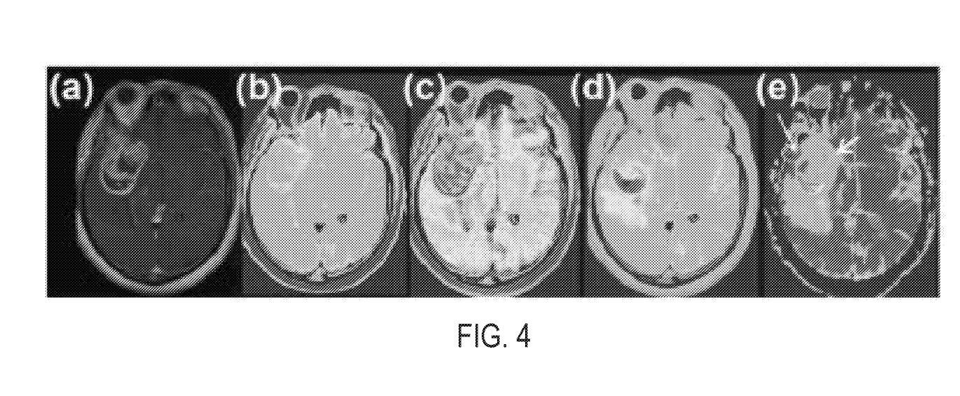

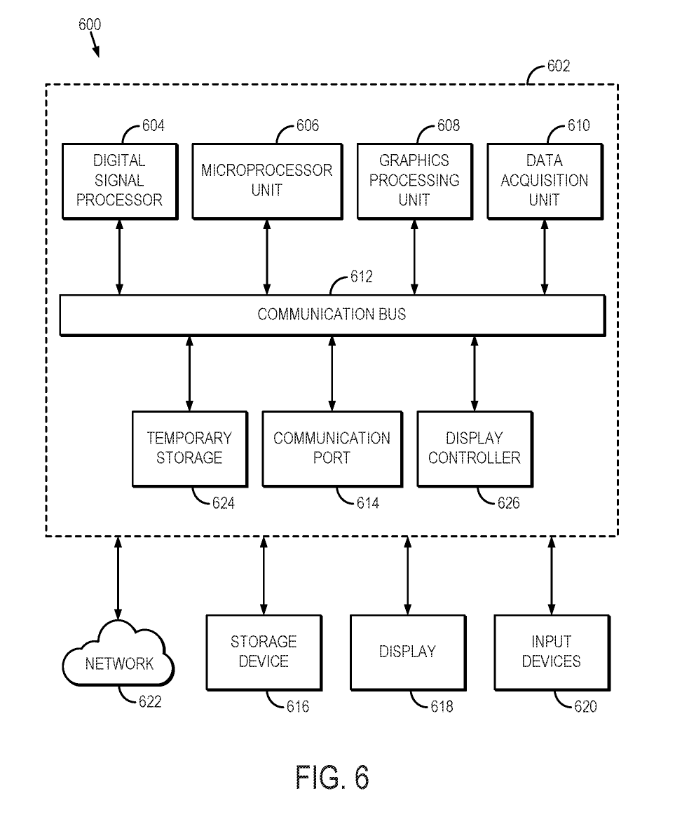

The variability of ADC values across patient before and after normalization are demonstrated in FIG. 3. A total of 21 patients were evaluated, the mean age was 58+/−13 years (10 men/11 women). Representative images of T1, T2, and ADC are shown in FIGS. 4A-4E. Note the intra-tumoral variability in ADC values that may reflect the presence of genetic phenotype expressing high and low cellularity. An example of a pMGMT predictive genomic profile map is illustrated in FIG. 5.

Mean values of normal appearing white matter, gray matter, and CSF showed no significant differences in patients with methylated and unmethylated tumors (P>0.05). However, the mean values from ROIs covering the tumors showed significant difference in pre-contrast T1, ADC, and the change in T1 resulting from contrast administration.

Discussion.

The example study described here has shown that appropriately normalized, quantitative MRI can improve the prediction of local genetic traits, specifically pMGMT status, in treatment naïve glioblastoma multiforme. In this technical development, methods for producing parametric color maps that allow direct visualization of the pattern of local genomic traits overlaid on any desired, co-registered MRI images have also been provided.

The normalized quantitative maps of T1, T1 post contrast, T2 and ADC shown in FIGS. 3A-3E represent the justification for this research. The parametric image of local genomic predictions overlaid on the tumor in FIG. 5 suggests a varied landscape of pMGMT status within the tumor. It is well known that some tumors may exhibit a multitude of different local phenotypes, which proposes that single or even multiple biopsies may not be fully representative of the whole tumor.

Thus, it may be that some portions of a given tumor may be resistant to a given therapy while others may be sensitive. While gross total resection of tumor is ideal, it is not always possible, and some tumors are simply inoperable. The utility of imaging-derived heat maps of genetic manifestations can be well appreciated in these situations, where genetic information is desired but cannot be obtained. Incomplete tumor resection is sometimes inevitable if tumor has commandeered eloquent cortex or white matter tracts. This same situation may pose risks to biopsy as well.

Noninvasive, reliable imaging approaches, such as those described here, could still survey tumors for clinically actionable genomic traits even in these precarious locations, and elegantly render that information for the radiologist. The model proposed here can be readily adapted into other, more sophisticated radiogenomic associations by employing machine learning models. Verhaak GBM classifications, IDH1 and IDH2 mutations, and other common or esoteric genetic targets in GBM or other tumors or pathologies can be investigated and implemented into a parameterized genomic heat map.

The methods described here can also be readily applied to radiogenomic investigations outside of the brain, and could be aimed at a wide variety of tumors in the body. One goal of the methods described here is to determine a genetic phenotype through the extraction of morphological features from tumors. Once the features are determined, advanced machine learning algorithms are use to create predictive models. The methods described here, however, assumes that multiple phenotypes exist within a tumor and a local, rather than global, analysis of the tumor is conducted.

Thus, methods for converting grayscale values into normalized estimations of physiological quantities have been provided. These methods provide consistency of data from scan-to-scan, which allows the data to be used for high-fidelity radiogenomic analyses. Imaging-derived radiogenomic heat maps, based on trained models of quantitative radiogenomic associations, provide a novel technique to survey the often varied genetic landscape within a tumor.

Referring now to FIG. 6, a block diagram of an example computer system 600 that can be configured to implement the methods for quantitative calibration and genomic profile map generation described here, is illustrated. Data, such as magnetic resonance images, can be provided to the computer system 600 from a data storage device, and these data are received in a processing unit 602.

In some embodiments, the processing unit 602 can include one or more processors. As an example, the processing unit 602 may include one or more of a digital signal processor (“DSP”) 604, a microprocessor unit (“MPU”) 606, and a graphics processing unit (“GPU”) 608. The processing unit 602 can also include a data acquisition unit 610 that is configured to electronically receive data to be processed. The DSP 604, MPU 606, GPU 608, and data acquisition unit 610 are all coupled to a communication bus 612. As an example, the communication bus 612 can be a group of wires, or a hardwire used for switching data between the peripherals or between any component in the processing unit 602.

The DSP 604 can be configured to implement the methods described here. The MPU 606 and GPU 608 can also be configured to implement the methods described here in conjunction with the DSP 604. As an example, the MPU 606 can be configured to control the operation of components in the processing unit 602 and can include instructions to implement the methods for calibrating qualitative magnetic resonance images to convert those images into quantitative maps of physiological parameters on the DSP 604. Also as an example, the GPU 608 can process image graphics, such as displaying magnetic resonance images, quantitatively calibrated magnetic resonance images, and genomic profile maps, whether alone or overlaid on magnetic resonance images.

The processing unit 602 preferably includes a communication port 614 in electronic communication with other devices, which may include a storage device 616, a display 618, and one or more input devices 620. Examples of an input device 620 include, but are not limited to, a keyboard, a mouse, and a touch screen through which a user can provide an input.

The storage device 616 is configured to store data, which may include magnetic resonance images, whether these data are provided to or processed by the processing unit 602. The display 618 is used to display images and other information, such as magnetic resonance images, quantitatively calibrated magnetic resonance images, and genomic profile maps, whether alone or overlaid on magnetic resonance images.

The processing unit 602 can also be in electronic communication with a network 622 to transmit and receive data and other information. The communication port 614 can also be coupled to the processing unit 602 through a switched central resource, for example the communication bus 612.

The processing unit 602 can also include a temporary storage 624 and a display controller 626. As an example, the temporary storage 624 can store temporary information. For instance, the temporary storage 624 can be a random access memory.

The present invention has been described in terms of one or more preferred embodiments, and it should be appreciated that many equivalents, alternatives, variations, and modifications, aside from those expressly stated, are possible and within the scope of the invention.

1. A method for producing quantitative maps that indicate quantitative values of physiological parameters from magnetic resonance images, the steps of the method comprising:
(a) providing to a computer system, a plurality of magnetic resonance images depicting a subject, the plurality of magnetic resonance images having different physical contrasts; and
(b) converting the plurality of magnetic resonance images with the computer system to produce quantitative physiological parameter maps by calibrating the magnetic resonance images based on signal models associated with how the magnetic resonance images were acquired.

2. The method as recited in claim 1, wherein step (b) includes computing average grayscale values in the magnetic resonance images and calibrating the average grayscale values based on signal models associated with how the respective magnetic resonance images were acquired.

3. The method as recited in claim 2, wherein calibrating the average grayscale values includes using a recursion analysis based on the signal models associated with how the respective magnetic resonance images were acquired.

4. The method as recited in claim 3, wherein calibrating the magnetic resonance images includes determining coefficients from the recursion analysis and applying the coefficients to the magnetic resonance images to convert grayscale values therein to quantitative physiological parameter values.

5. The method as recited in claim 3, wherein the recursion analysis includes fitting the average grayscale values to linear approximations of the signal models.

6. The method as recited in claim 1, further comprising:
(c) determining with the computer system, a likelihood of a particular gene expression in a tissue in the subject based on the quantitative physiological parameter maps and a trained model that relates genetic information to quantitative physiological parameters; and
(d) producing with the computer system, a genomic profile map that indicates a level of genetic expression for the particular gene using the determined likelihood of the particular gene expression in the tissue.

7. The method as recited in claim 6, wherein step (c) includes calculating statistics of the quantitative physiological parameter maps produced in step (b) and determining the likelihood of the particular gene expression in the tissue in the subject based on the calculated statistics.

8. The method as recited in claim 7, wherein the calculated statistics are compared with statistics based on genetic information provided to the computer system.

9. The method as recited in claim 8, wherein comparing the calculated statistics with the statistics based on the provided genetic information includes using a machine learning algorithm.

10. The method as recited in claim 6, wherein step (d) includes using a statistical test of diagnostic accuracy to relate the likelihood of a particular gene expression in a tissue in the subject to values in the genomic profile map.

11. The method as recited in claim 10, wherein the statistical test of diagnostic accuracy includes at least one of receiver operator characteristics (ROC) analysis, a student's T-test, or a chi-squared test.

12. The method as recited in claim 10, wherein relating the likelihood of a particular gene expression in a tissue in the subject to values in the genomic profile map includes calculating local variations in the quantitative physiological parameter maps from locations having statistically significant likelihood of a particular gene expression in a tissue in the subject, and converting the local variations in the quantitative physiological parameter maps to genomic profile map values based on an ROC curve.

13. The method as recited in claim 12, wherein converting the local variations in the quantitative physiological parameter maps to genomic profile map values includes extracting a true positive fraction from the ROC curve as a function of the local variations in the quantitative physiological parameter maps.

14. The method as recited in claim 1, wherein step (b) includes selecting at least one region-of-interest in the magnetic resonance images and calibrating the magnetic resonance images based on image intensity values in the at least one region-of-interest.

15. The method as recited in claim 1, wherein step (b) includes segmenting the magnetic resonance images into at least one different tissue type and calibrating the magnetic resonance images based on image intensity values in the segmented at least one different tissue type.
